(19) United States
(12) Patent Application Publication
(10) Pub. No.: US 2002/0058025 A1
(43) Pub. Date: May 16, 2002

(54) Stromal cell use

(76) Inventors: Darwin J. Prockop, Philadelphia, PA (US); Russell G. Reiss, Salt Lake City, UT (US); John Langell, San Jose, CA (US)

Correspondence Address: KATHRYN DOYLE, PH.D., J.D., MORGAN, LEWIS & BOCKIUS, L.L.P., 1701 Market Street, Philadelphia, PA, 19103-2921, US

(21) Appl. No.: 09/839,711

(22) Filed: Apr. 20, 2001

Related U.S. Application Data
(63) Continuation of application No. PCT/US99/25134, filed on Oct. 26, 1999.
(60) Provisional application No. 60/105,671, filed on Oct. 26, 1998.

(51) Int. Cl. A61K 45/00; C12N 5/08

(52) U.S. Cl. 424/93.71; 435/372

(57) ABSTRACT

The invention relates to the use of marrow stromal cells to enhance hematopoiesis in a mammal.

### FIELD OF THE INVENTION

The field of the invention is use of marrow stromal cells in enhancing hematopoiesis.

### BACKGROUND OF THE INVENTION

In addition to the hematopoietic stem cells (HSC), bone marrow contains stem-like precursors for non-hematopoietic cells, such as osteoblasts, chondrocytes, adipocytes and myoblasts (Owen et al., 1988, In: Cell and Molecular Biology of Vertebrate Hard Tissues, pp. 42-60, Ciba Foundation Symposium 136, Chichester, UK; Caplan, 1991, J. Orthop. Res. 9:641-650; Prockop, 1997, Science 276:71-74). Non-hematopoietic precursors of the bone marrow have been variously referred to as colony-forming-units-fibroblasts, mesenchymal stem cells, stromal cells, and marrow stromal cells (MSCs).

MSCs are mesenchymal precursor cells (Friedenstein et al., 1976, Exp. Hemat. 4:267-274) that are characterized by their adherence properties when bone marrow cells are removed from a mammal and are transferred to plastic dishes. Within about four hours, stromal cells adhere to the plastic and can thus be isolated by removing non-adherent cells from the dishes. Bone marrow cells that tightly adhere to plastic have been studied extensively (Castro-Malaspina et al., 1980, Blood 56:289-301; Piersma et al., 1985, Exp. Hematol. 13:237-243; Simmons et al., 1991, Blood 78:55-62; Beresford et al., 1992, J. Cell. Sci. 102:341-351; Liesveld et al., 1989, Blood 73:1794-1800; Liesveld et al., 1990, Exp. Hematol. 19:63-70; Bennett et al., 1991, J. Cell. Sci. 99:131-139).

Stromal cells are believed to participate in the creation of the microenvironment within the bone marrow in vivo. When isolated, stromal cells are initially quiescent but eventually begin dividing so that they can be cultured in vitro. Expanded numbers of stromal cells can be established and maintained. Stromal cells have been used to generate colonies of fibroblastic adipocytic and osteogenic cells when cultured under appropriate conditions. If the adherent cells are cultured in the presence of hydrocortisone or other selective conditions, populations enriched for hematopoietic precursors or osteogenic cells are obtained (Carter et al., 1992, Blood 79:356-364 and Bienzle et al., 1994, Proc. Natl. Acad. Sci. USA 91:350-354).

There are several examples of the use of stromal cells. European Patent EP 0,381,490, discloses gene therapy using stromal cells. In particular, a method of treating hemophilia is disclosed. Stromal cells have been used to produce fibrous tissue, bone or cartilage when implanted into selective tissues in vivo (Ohgushi et al., 1989, Acta Orthop. Scand. 60:334-339; Nakahara et al., 1992, J. Orthop. Res. 9:465-476; Niedzwiedski et al., 1993, Biomaterials 14:115-121; and Wakitani et al., 1994, J. Bone & Surg. 76A:579-592). In some reports, stromal cells were used to generate bone or cartilage in vivo when implanted subcutaneously with a porous ceramic (Ohgushi, et al., 1989, Acta. Orthop. Scand. 60:334-339), intraperitoneally in a difflusion chamber (Nakahara et al., 1991, J. Orthop. Res. 9:465-476), percutaneously into a surgically induced bone defect (Niedzwiedski et al., 1993, Biomaterials 14:115-121), or transplanted within a collagen gel to repair a surgical defect in a joint cartilage (Wakitani et al., 1994, J. Bone Surg. 76A: 579-592). Piersma et al. (1983, Brit. J. Hematol. 94:285-290), disclose that after intravenous bone marrow transplantation, the fibroblast colony-forming cells which make up the hemopoietic stroma lodge and remain in the host bone marrow. Stewart et al. (1993, Blood 81:2566-2571), recently observed that unusually large and repeated administrations of whole marrow cells produced long-term engrafiment of hematopoietic precursors into mice that had not undergone marrow ablation. Also, Bienzle et al. (1994, Proc. Natl. Acad. Sci. USA 91:350-354), successfully used long-term bone marrow cultures as donor cells to permanently populate hematopoietic cells in dogs without marrow ablation. In some reports, stromal cells were used either as cells that established a microenvironment for the culture of hematopoietic precursors (Anklesaria, 1987, Proc. Natl. Acad. Sci. USA 84:7681-7685) or as a source of an enriched population of hematopoietic stem cells (Kiefer, 1991, Blood 78:2577-2582).

There is a long-felt and acute need for methods for enhancing recovery of hematopoiesis in mammals having ablated marrow. The present invention meets this need.

### SUMMARY OF THE INVENTION

The invention relates to a method of rescuing a mammal from a lethal dose of total body irradiation. The method comprises administering marrow stromal cells from an allogenic but otherwise identical donor mammal to an irradiated mammal, thereby rescuing the mammal from a lethal dose of total body irradiation.

In one aspect, the mammal is selected from the group consisting of a rodent, a horse, a cow, a pig, a dog, a cat, a non-human primate, and a human. In another aspect, the mammal is a human.

In another aspect, the administration is infusion.

The invention also includes a method of enhancing hematopoiesis in a mammal. The method comprises administering marrow stromal cells from an allogenic but otherwise identical donor mammal to a mammal, thereby enhancing hematopoiesis in the mammal.

In one aspect, the mammal is selected from the group consisting of a rodent, a horse, a cow, a pig, a dog, a cat, a non-human primate, and a human. In another aspect, the mammal is a human.

In another aspect, the administration is infusion.

In addition, there is provided a method of enhancing hematopoietic stem cell differentiation in a mammal given a lethal dose of total body irradiation. The method comprises administering marrow stromal cells from an allogenic but otherwise identical donor mammal to an irradiated mammal, thereby enhancing hematopoietic stem cell differentiation in the mammal.

In one aspect, the mammal is selected from the group consisting of a rodent, a horse, a cow, a pig, a dog, a cat, a non-human primate, and a human. In another aspect, the mammal is a human.

In another aspect, the administration is infusion.

Also included in the invention is a method of enhancing the hematopoietic recovery in a mammal given a lethal dose of total body irradiation. The method comprises administering marrow stromal cells from an allogenic but otherwise identical donor mammal to an irradiated mammal, thereby enhancing the hematopoietic recovery in said mammal.

A method of treating a mammal comprising an ablated marrow is also included in the invention. The method comprises administering marrow stromal cells from an allogenic but otherwise identical donor mammal to a mammal, thereby treating the mammal comprising an ablated marrow.

The invention also includes a method of enhancing hematopoiesis in a mammal comprising an ablated marrow. The method comprises administering marrow stromal cells from an allogenic but otherwise identical donor mammal to a mammal, thereby enhancing hematopoiesis in the mammal comprising an ablated marrow.

The invention includes a method of increasing the survival of a mammal exposed to a lethal dose of total body irradiation. The method comprises administering marrow stromal cells from an allogenic but otherwise identical donor mammal to an irradiated mammal, thereby increasing the survival of a mammal exposed to a lethal dose of total body irradiation.

### BRIEF DESCRIPTIONS OF THE DRAWINGS

FIG. 1A is a graph depicting the recovery of hematopoiesis in rats irradiated and infused with allogenic MSCs compared with nonirradiated control animals which did not receive any cells. The graph depicts a rise in hematocrit in irradiated rats (▪) over time compared with control rats (♦).

FIG. 1B is a graph depicting the recovery of hematopoiesis in rats irradiated and infused with allogenic MSCs compared with nonirradiated control animals which did not receive any cells. The graph depicts a rise in white blood cells (expressed in thousands per μl) in irradiated rats (▪) over time compared with control rats(♦).

FIG. 2A is a graph depicting the FACS profile of a mixed population of PBLs from Wistar Furth rats (WF) and Lewis (LEW) rats stained using an FITC-conjugated mAb (RTA^{a,b,l}) for MHC-I.

FIG. 2B is a graph depicting the FACS profile of PBLs from Wistar Furth rats (WF) previously infused with MSCs from Lewis (LEW) rats stained using an FITC-conjugated mAb (RTA^{a,b,l}) for MHC-I demonstrating that PBLs in recipient WF are of endogenous origin and they are not derived from the LEW cells.

FIG. 3A is a graph depicting the amplification plots of real time PCR assays demonstrating the threshold cycles for each dilution of male Lewis (LEW) rat DNA in female WF rat DNA. The amount of male LEW rat DNA in 1 μg of WF female rat DNA is expressed by percentages as follows: (a) 100%, (b) 10%, (c) 1%, (d) 0.1%, (e) 0.01%, (f) 0.001%, and (g) control with 0%.

FIG. 3B is a standard curve based on the threshold cycle data for the amplification plots of the six dilution standards depicted in FIG. 3A. Based upon this standard curve, the amount of male LEW rat DNA in a sample also containing WF female rat DNA may be calculated by determining the threshold cycle using real time PCR.

### DETAILED DESCRIPTION OF THE INVENTION

The invention is based on the discovery that rats receiving a lethal, but not myloablative, dose of total body irradiation (TBI) may be rescued by the intraperitoneal injection of allogenic marrow stromal cells administered shortly after the irradiation. The allogenic MSCs enhance the recovery of hematopoiesis in recipient animals. However, the circulating PBLs in rescued animals were not derived from the donor animals as demonstrated by the fact that the cells express the endogenous MHC Class II antigens of the recipient and do not express the Class I MHC antigens of the donor. Further, highly sensitive real time PCR-based assays capable of detecting as little as 10 ng of donor male LEW rat Y-chromosome specific DNA in 1 μg of recipient female WF DNA did not detect the presence of male LEW rat DNA in samples of genomic DNA obtained from various tissues from the bodies of recipient animals. Further, animals irradiated with a myloablative dose of TBI were not rescued by administration of donor MSCs. These results demonstrate that the donor MSCs can rescue animals from lethal doses of radiation by enhancing the hematopoietic recovery of the animal's own hematopoietic stem cells (HSC) which have not been eliminated by the radiation.

Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, “stromal cells”, “marrow stromal cells,” “adherent cells,” and “MSCs” are used interchangeably and meant to refer to the small fraction of cells in bone marrow which can serve as stem-cell-like precursors of osteocytes, chondrocytes, and adipocytes, and the like, which can be isolated from bone marrow by their ability to adhere to plastic dishes. Marrow stromal cells may be derived from any animal. In some embodiments, stromal cells are derived from rodents, preferably rats. However, the invention is not limited to rodent MSCs; rather, the invention encompasses mammalian, more preferably human, marrow stromal cells.

By the term “ablated marrow” as that term is used herein, is meant that the marrow is not capable of hematopoiesis but is not completely devoid of hematopoietic stem cells capable of growth and differentiation. Ablation may be caused by irradiation, chemotherapeutics, or any other method which ablates hematopoiesis.

By the term “lethal dose total body irradiation,” as the term is used herein, is meant total body irradiation which in not myloablative but which otherwise kills over 50% of the animals irradiated.

In one preferred embodiment, the lethal dose in rats was determined to be 900 cGy of total body irradiation. However, one skilled in the art would appreciate that the lethal radiation dose for any animal would vary depending on various factors including the size, age, and physical condition of the animal, and the like. Accordingly, the present invention should not be construed as being limited to any particular lethal dose; rather, a wide range of lethal doses is encompassed in the invention.

By the term “myloablative,” as that term is used herein, is meant that the treatment destroy all or a substantial portion of the hematopoietic stem cells such that endogenous hematopoiesis cannot be restored by any method or treatment.

The term “endogenous hematopoiesis,” as used herein, is intended to mean the production of peripheral blood lymphocytes derived from the animal's own hematopoietic stem cells.

In one preferred embodiment, endogenous hematopoiesis was detected by fluorescence activated cell sorter analysis of the MHC antigens expressed on the PBLs of an animal. In another preferred embodiment, the lack of exogenous DNA from a marrow stromal cell donor animal was confirmed by real time PCR using probes and primer specific for the donor DNA, e.g., male rat Y-chromosome-specific DNA. The present invention should not, however, be limited to these methods of detecting the origin of the PBLs to confirm the endogenous nature of the observed hematopoiesis. Further, the invention is not limited to the specific MHC antibodies or the specific primer pairs or probes disclosed. Rather, the invention encompasses other methods currently known to the art or to be developed for ascertaining the origin of the hematopoietic cells in an animal.

By the term “enhancing the hematopoietic recovery,” as the term is used herein, is meant any increase in the hematopoiesis detected in an animal caused by a treatment compared to the hematopoiesis in the animal before the treatment or in an otherwise identical but untreated animal.

By the term “treating a mammal comprising an ablated marrow,” as the term is used herein, is meant increasing the endogenous hematopoiesis in an animal by any method compared with the animal before treatment or with an otherwise identical animal which is not treated. The increase in endogenous hematopoiesis can be assessed using the methods disclosed herein or any other method for assessing endogenous hematopoiesis in an animal.

The term “rescuing a mammal from a lethal dose of total body irradiation,” as used herein, means increasing the endogenous hematopoiesis in an animal exposed to a lethal dose of total body irradiation by any treatment compared with the endogenous hematopoiesis in the animal before treatment or with a the endogenous hematopoiesis in an otherwise identical animal which is not treated. The increase in endogenous hematopoiesis can be assessed using the methods disclosed herein or any other method for assessing endogenous hematopoiesis in an animal.

By the term “increasing the survival of a mammal exposed to a lethal dose of total body irradiation,” as the term is used herein, is meant increasing the period of time that a mammal survives following exposure to a lethal dose of total body irradiation. The length of time of survival post-irradiation can be measured and any significant increase in survival time can be determined using standard statistical analysis methods as disclosed herein or as are well-known in the art such that a method that increases the survival of an irradiated mammal compared with the length of survival of an otherwise identical mammal that is not treated can be determined.

Description

The invention includes a method of rescuing a mammal from a lethal dose of total body irradiation. The method comprises administering marrow stromal cells from an allogenic but otherwise identical donor mammal to an irradiated mammal, thereby rescuing the mammal from a lethal dose of total body irradiation. The invention is based on the novel discovery disclosed herein that administering MSCs to an irradiated animal, where the radiation dose is not myloablative, mediates the endogenous repopulation of the mammal's hematopoietic system.

In a preferred embodiment, five million MSCs were administered intraperitoneally by injection into rats. However, the invention is not limited to this method of administering the cells or to any particular number of cells. Rather, the cells may be administered to (e.g., introduced into) the animal by any means, including intravenous transfusion and the like. Further, the number of MSCs to be administered will vary according to the animal being treated and the appropriate number of MSCs can be easily determined for that animal by methods well known in the art of using stromal cells to affect hematopoiesis as discussed in the above-cited references and as disclosed elsewhere herein.

After isolating the stromal cells, the cells can be administered to a mammal, preferably a human, upon isolation or following a period of in vitro culture. Isolated stromal cells may be administered upon isolation, or may be administered within about one hour after isolation. Generally, marrow stromal cells may be administered immediately upon isolation in situations in which the donor is large and the recipient is small (e.g., an infant). It is preferred that stromal cells are cultured prior to administration. Isolated stromal cells can be cultured from 1 hour to up to over a year. In some preferred embodiments, the isolated stromal cells are cultured prior to administration for a period of time sufficient to allow them to convert from non-cycling to replicating cells. In some embodiments, the isolated stromal cells are cultured for 3-30 days, preferably, 5-14 days, more preferably, 7-10 days. In other embodiments, the isolated stromal cells are cultured for 4 weeks to a year, preferably, 6 weeks to 10 months, more preferably, 3-6 months.

It is preferred that stromal cells are cultured prior to administration. Isolated stromal cells can be cultured for 3-30 days, in some embodiments, 5-14 days, in other embodiments, 7-10 days prior to administration. In some embodiments, the isolated stromal cells are cultured for 4 weeks to a year, in some embodiments, 6 weeks to 10 months, in some embodiments, 3-6 months prior to administration.

For administration of stromal cells to a human, the isolated stromal cells are removed from culture dishes, washed with saline, centrifuged to a pellet and resuspended in a glucose solution which is infused into the patient. In some embodiments, bone marrow ablation, but not myloablation, is undertaken prior to administration of MSCs. The immune responses suppressed by agents such as cyclosporin must also be considered. Bone marrow ablation may be accomplished by X-radiating the individual to be treated, administering drugs such as cyclophosphamide or by a combination of X-radiation and drug administration. In some embodiments, bone marrow ablation is produced by administration of radioisotopes known to kill metastatic bone cells such as, for example, radioactive strontium, ¹³⁵Samarium or ¹⁶⁶Holmium (see Applebaum et al., 1992, Blood 80(6):1608-1613).

Between about 10⁵ and about 10¹³ marrow stromal cells per 100 kg body weight are administered per infusion. In some embodiments, between about 1.5×10⁶ and about 1.5×10¹² cells are infused intravenously per 100 kg body weight. In some embodiments, between about 1×10⁹ and about 5×10¹¹ cells are infused intravenously per 100 kg body weight. In some embodiments, between about 4×10⁹ and about 2×10¹¹ cells are infused per 100 kg body weight. In some embodiments, between about 5×10⁸ cells and about 1×10¹ cells are infused per 100 kg body weight.

In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days.

In some embodiments, a single administration of between about 1 and about 10¹³ cells per 100 kg body weight is provided. In some embodiments, a single administration of between about 1.5×10⁸ and about 1.5×10¹² cells per 100 kg body weight is provided. In some embodiments, a single administration of between about 1×10⁹ and about 5×10¹¹ cells per 100 kg body weight is provided. In some embodiments, a single administration of about 5×10¹⁰ cells per 100 kg body weight is provided. In some embodiments, a single administration of 1×10¹⁰ cells per 100 kg body weight is provided.

In some embodiments, multiple administrations of between about 10⁵ and about 10¹³ cells per 100 kg body weight are provided. In some embodiments, multiple administrations of between about 1.5×10⁸ and about 1.5×10¹² cells per 100 kg body weight are provided. In some embodiments, multiple administrations of between about 1×10⁹ and about 5×10¹¹ cells per 100 kg body weight are provided over the course of 3-7 consecutive days. In some embodiments, multiple administrations of about 4×10⁹ cells per 100 kg body weight are provided over the course of 3-7 consecutive days. In some embodiments, multiple administrations of about 2×10¹¹ cells per 100 kg body weight are provided over the course of 3-7 consecutive days.

In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

Further, the invention includes a method of enhancing hematopoiesis in a mammal. The method comprises administering marrow stromal cells from an allogenic but otherwise identical donor mammal to a mammal, thereby enhancing hematopoiesis in the mammal. One skilled in the art would appreciate, based upon the disclosure provided herein, that hematopoiesis is enhanced in the mammal because, as disclosed herein, administration of MSCs to a mammal mediates the endogenous hemopoietic reconstitution of the animal.

One skilled in the art would appreciate, based upon the disclosure provided herein, that an individual suffering from a disease, disorder, or a condition that is characterized by or mediated through an inhibition or decrease in hematopoiesis can be treated by administration of MSCs to enhance hematopoiesis in the individual.

The invention includes a method of enhancing hematopoietic stem cell differentiation in a mammal given a lethal dose of total body irradiation. The method comprising administering marrow stromal cells from an allogenic but otherwise identical donor mammal to an irradiated mammal, thereby enhancing hematopoietic stem cell differentiation in the mammal. The method is based on the novel discovery disclosed herein that administration of MSCs to a mammal following exposure to a lethal dose of total body irradiation mediates endogenous hemopoietic reconstitution in the mammal. Such reconstitution necessarily involves the differentiation of endogenous hemopoietic stem cells, and the like, to proliferate and differentiate into the various hemopoietic cell types. Thus, administration of MSCs which mediates endogenous hemopoietic reconstitution necessarily involves enhancing hemopoietic stem cell differentiation involved in such reconstitution.

The invention also includes a method of enhancing the hematopoietic recovery in a mammal given a lethal dose of total body irradiation. The method comprises administering marrow stromal cells from an allogenic but otherwise identical donor mammal to an irradiated mammal, thereby enhancing the hematopoietic recovery in the mammal.

A person skilled in the art would appreciate, based upon the disclosure provided herein, that administration of MSCs which mediates endogenous hematopoietic reconstitution in a mammal enhances hematopoietic recovery in the mammal. That is, administration of MSCs mediates repopulation of the mammal's hematopoietic system thus enhancing hematopoietic recovery in the mammal.

The invention includes a method of treating a mammal comprising an ablated marrow. The method comprises administering marrow stromal cells from an allogenic but otherwise identical donor mammal to a mammal, thereby treating the mammal comprising an ablated marrow. This is because, as disclosed herein, administering MSCs to a mammal causes hematopoietic reconstitution, or, at the very least, an increase in endogenous hematopoiesis, in the mammal thereby treating the radiation-induced decrease of hematopoietic cells in the mammal due to marrow ablation.

The invention further includes a method of enhancing hematopoiesis in a mammal comprising an ablated marrow. The method comprises infusing marrow stromal cells from an allogenic but otherwise identical donor mammal into a mammal, thereby enhancing hematopoiesis in the mammal comprising an ablated marrow. The method is based on the data disclosed herein demonstrating, for the first time, that administration of MSCs to a mammal comprising ablated bone marrow mediates the endogenous reconstitution of the mammal's own hematopoiesis. Thus, administration of MSCs enhances hematopoiesis required for reconstitution of the mammal as demonstrated herein.

The invention includes a method of increasing survival of a mammal exposed to a lethal dose of total body irradiation. The method comprises administering marrow stromal cells from an allogenic but otherwise identical donor mammal to an irradiated mammal, thereby increasing the survival of a mammal exposed to a lethal dose of total body irradiation. One skilled in the art would appreciate, based upon the disclosure provided herein, that survival of exposure to a lethal dose of TBI is dependent, at least in part, on the hematopoietic reconstitution of the mammal. The data disclosed herein demonstrate that hematopoietic reconstitution is mediated by administration of MSCs to a mammal following exposure to a lethal dose of TBI. Further, the data demonstrate that the survival, as measured by increased number of animals surviving after exposure, was greatly increased by administration of MSCs to the animals compared with otherwise identical animals which were irradiated but to which no MSCs were administered. Thus, one skilled in the art would appreciate based on the instant disclosure, that survival of exposure to a lethal dose of TBI by a mammal is significantly increased by administration of MSCs to the mammal which MSCs mediate enhanced hematopoiesis which is necessary for survival from otherwise lethal irradiation.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

Allogenic Rat Marrow Stromal Cells Enhance Survival and Recovery of Endogenous Hematopoiesis Following Lethal Irradiation

The experiments presented in this example may be summarized as follows.

The data disclosed herein demonstrate that the engrafiment of marrow stromal cells (MSC) across a full MHC Class I and Class II barrier can rescue recipient animals from lethal total body irradiation (TBI) with only a single intraperitoneal (i.p.) injection of 5×10⁶ allogenic MSCs. Ten week old male Lewis (LEW) rats were used as MSC donors and ten week old female Wistar Furth (WF) rats were used as recipients. Whole bone marrow was harvested from the femurs and tibias of LEW rats and the cells were plated into plastic culture flasks. At day 3 post-harvest, all unattached cells and media were removed leaving the adherent cell layer, and fresh media was added to the flasks. The cells were passaged by trypsinization and the cultures were maintained until the end of second passage with media changed twice weekly. Thirty-one WF female rats received a lethal dose of 900 cGy TBI and i.p. injection of 5×10⁶ LEW MSCs four hours after irradiation. Twenty-two WF female rats received 900 cGy TBI alone and served as controls. All 22 animals in the control group expired with a mean survival of 15 days. In contrast, 21 of 31 rats in the experimental group recovered entirely from the TBI with no gross or histologic evidence of graft versus host disease (GVHD). Allogenic MSC transplantation was repeated at a higher radiation dose of 1000 cGy TBI thought to be myloablative. Animals irradiated with 1000 cGy TBI (n=12 in each group) had no survivors with mean survival of 8.8 days and 9.0 days for treated and control groups, respectively.

Peripheral blood from all survivors of 900 cGy TBI was flow sorted using FITC directly labeled monoclonal antibodies specific for donor MHC class I. At 30 days after MSC transplantation, there was no evidence of donor hemopoietic repopulation, suggesting that survival and hematopoietic recovery was not due to donor hemopoietic stem cell (HSC) contamination. These results demonstrate that allogenic MSCs can provide rescue to animals receiving lethal but not myloablative TBI. Without wishing to be bound by any particular theory, these data suggest that allogenic MSCs in these experiments are providing support for endogenous HSCs that have not been eliminated by lethal conditioning.

The Materials and Methods used in the experiments presented in this example are now described.

Animals

Eight week old Lewis and Wistar Furth rats were obtained from Haran Sprague-Dawley Company, Indianapolis, Ind. All animals were acquired without viral infestation and kept in an environment free of virus in the animal facility at Allegheny University of the Health Sciences. All animals were handled in accord with the “Principles of Laboratory Animal Care” formulated by the National Society for Medical Research and the “Guide for the Care and Use of Laboratory Animals” prepared by the National Institutes of Health (NIH Publication No. 86-23, revised 1985).

Bone Marrow Stromal Cell Cultures

Eight week old male Lewis rats were euthanized with a 70% CO₂/30% O₂ gas mixture. Animals were then shaved and prepped with alcohol and provodine solution. The long bones of the lower extremity were harvested and kept in ice cold cell culture media (DMEM, Sigma Chemical Co., St. Louis, Mo.) containing 10% fetal calf serum (FCS), penicillin/streptomycin, and Amphotericin B. Under sterile conditions, a 21 gauge needle containing culture media was used to flush marrow from the tibias and femurs. Whole bone marrow was then dispersed using a 10 ml pipette. A 25 ml final volume of marrow-containing media was added to a sterile T-75 (Falcon) plastic culture flask and incubated at 37° for 3 days. After 3 days, the entire nonadherent layer was discarded and fresh media was added to the flasks. The adherent stromal cell layer was then allowed to expand to 80% confluence prior the splitting with trypsin. The media was changed twice weekly. The cells used for transplantation were allowed to reach third passage.

Bone Marrow Stromal Cell Transplantation

Recipients were 10 week old female WF rats. Prior to MSC injection, the animals received either 1000, 900, 500 or 0 cGy total body X irradiation (TBI) in a single dose from a linear accelerator maintained at Allegheny University of the Health Sciences (Philadelphia, Pa.) (AUHS). MSC grown to third passage in culture were washed twice with sterile phosphate buffered saline (PBS) and lifted from plastic culture flasks by trypsinization. The cells were washed twice in serum-free media and then resuspended in sterile serum-free media at a final concentration of 5×10⁶ cells per ml. Cell viability was confirmed by trypan blue exclusion assay and the cells were counted using a hemocytometer. Recipient animals received a single 1 ml i.p. injection containing 5×10⁶ MSC within 4 hours of receiving a single dose of TBI. Control animals received TBI and i.p. injection with 1 ml of sterile serum-free media No MSC were administered to control groups. In cases where animals succumbed, survival was measured in days from time of transplantation to death.

Irradiated MSC

MSC were prepared as previously described elsewhere herein. Fifty million cells were resuspended in 50 ml of serum free media and exposed to 10,000 cGy from a ¹³⁷Cs irradiator. The irradiated cells were then washed twice and resuspended in sterile serum-free media prior to i.p. injection.

Peripheral Blood Count

Five hundred microliters of whole peripheral blood were collected into pediatric complete blood count (CBC) vacutainer tubes containing EDTA. CBC, including hemoglobin and hematocrit, was performed by the clinical hematology laboratory at AUHS. A manual leukocyte count and differential was also performed on each sample.

Flow Cytometry

Peripheral blood lymphocytes (PBL) were stained with RTA^{a,b,l} FITC conjugated monoclonal antibody (mAb) for LEW (RTA¹) and RTA^{u} FITC conjugated polyclonal antibody serum for WF (RTA^{u}) for analysis by a fluorescence activated cell sorter (FACS). The cells were also stained with an irrelevant FITC-conjugated antibody isotype control. Briefly, 500 μl of peripheral blood were collected into heparinized 1.5 ml Eppendorf tubes by tail bleeding. The peripheral blood was transferred to 15 ml polypropylene tubes and PBL were isolated using a Ficoll hypaque centrifugation gradient. The buffy coat containing the PBL was washed twice in PBS and resuspended in FACS media. The cells were incubated on wet ice in the presence of donor and recipient specific antibodies for 30 minutes in the dark. Following incubation, the stained cells were again washed twice with FACS media and fixed with a 1% paraformaldehyde solution. Antibody-stained cells were then fluorescent antibody cell sorted using a Becton-Dickson (Lincoln Park, N.J.) FACScan. Data was analyzed using the Cell Quest software package provided by the manufacturer.

Preparation of Donor DNA Samples

Recipient animals were sacrificed and portal blood, liver, spleen, thymus, muscle, skin, bone marrow, and bone were harvested. Genomic DNA was purified from portal blood using DNAzol BD® (Gibco, Life Technologies) according to the manufacturer's protocol. Solid tissues were snap-frozen in liquid nitrogen immediately after harvest. Genomic DNA was prepared by grinding frozen tissue in a sterile mortar and pestle and digesting the dispersed tissue overnight in 20 mg/ml Proteinase K in the presence of 1% Sarkosyl and 0.5 mM EDTA at 55° C. DNA was purified from digests by standard phenol-chloroform extraction and ice-cold ethanol precipitation. The concentration of DNA was determined by 260/280 spectrophotometry.

Fluorescent Readout Real Time PCR of Genomic DNA

A custom designed pair of oligonucleotide primers amplifying a target sequence specific to the rat Y-chromosome and an oligonucleotide reporter “Taqman” type probe bearing the fluorescent molecule, 6-carboxy-fluorescein (FAM), at the 5′ end and the quencher molecule, 6-carboxy-tetramethyl-rhodamine (TAMRA), at the 3′ end were obtained from Perkin Elmer (Foster City, Calif.). Fluorescent readout “real time” quantitative sequence detection (QSD) polymerase chain reaction (PCR) of DNA samples was performed using an ABI Prism Model 7700 Sequence Detection System (Perkin Elmer, Foster City, Calif.).

The PCR mixture contained 1 μg genomic of DNA, 0.05 U/μl AmpliTaq Gold™ (Perkin Elmer), 0.01 U/μl AmpErase UNG™ (Perkin Elmer), 5.5 mM MgCl₂, 200 μM dATP, dCTP, dGTP, and 400 μM dUTP, 200 nM forward primer, 200 nM reverse primer, 100 μM TaqMan™ oligonucleotide probe, 1X TaqMan™ Buffer (Perkin Elmer) and q.s.d.H₂O for a final reaction volume of 50 μl/well. The PCR mix containing DNA was loaded into 96 well plates and sealed with optical caps. The thermocycling conditions were as follows: 94° C. for 10 minutes followed by 35 cycles of 94° C. for 15 seconds, 63° C. for 1 minute. Standard dilutions from 1:0 to 1:100,000 of male-to-female rat DNA were loaded in triplicate on each 96 well plate along with experimental samples to serve as reference standards used to prepare a standard curve. Real time PCR data was analyzed using the ABI Model 7700 software provided by the manufacturer.

Graft Verses Host Disease

Animals were monitored daily for signs of graft versus host disease (GVHD). This included examination for scaling dermis, swollen foot pads, anorexia, diarrhea, and weight loss. Upon sacrifice, the spleens were weighed and portions of the small bowel and the tongue were fixed in 10% buffered formalin, embedded in paraffin, and sectioned. Tissue staining was carried out with hematoxylin and eosin and the stained sections were examined by light microscopy for microscopic evidence of GVHD.

Cardiac Transplantation

Eight-week-old female LEW rats were used as cardiac donors. All operations were performed under general anesthesia. LEW donor hearts were harvested under cold arrest with ice slush. The vena cavae and pulmonary veins were ligated with 4.0 silk suture and the aorta and pulmonary artery were transacted using a fine scissors. Heterotopic cardiac transplantation was performed using the modified technique of Ono and Lindsey. The donor aorta and pulmonary artery were anastomosed to recipient abdominal aorta and inferior vena cava, respectively. Anastomoses were performed in an end-to-side fashion using 9.0 polypropylene monofilament suture. Transplant viability was determined by daily palpation of the recipient abdomen. If palpation was indeterminate, the graft was inspected under direct vision. Rejection was marked by the complete absence of ventricular contractions and confirmed histologically. Animals in which technical error lead to immediate graft failure or death were not included in the graft survival statistics.

The Results of the experiments presented in this example are now described.

Marrow Stromal Cells Enhance the Survival of the Lethally Irradiated Host with Only a Single i.p. Infection of 5×10⁶ MSC

Survival from lethal irradiation depends on the return of the hematogenous system. It is known that within the microenvironment of the bone marrow-a very complex relationship takes place between MSC and hemopoietic stem cells (HSC). In vitro, HSC have been shown to rely on MSC layers to survive as long term cultures. However, the in vivo relationship is still undefined despite numerous reports of hemopoietic rescue with subpopulations of HSC and other cells that may facilitate this recovery. The data disclosed herein demonstrate that MSC grown in culture until the third passage (approximately 5 weeks) not only enhanced the in vivo recovery of hematopoiesis but allowed complete recovery in the majority of the experimental group of animals that received a lethal dose of 900 cGy X-irradiation followed by a single intraperitoneal injection of MSCs (Table 1). Furthermore, animals that survived this treatment regimen exhibited no manifestations of graft verses host disease (GVHD). More specifically, twenty-one of thirty-one Wistar Furth (WF) female rats that received 900 cGy+1 ml of serum-free media containing 5×10⁶ MSC via intraperitoneal injection survived to a complete recovery. All 22 of the control animals received 900 cGy and identical i.p. injection of 1 ml of serum-media without the MSC component. None of the control group animals survived with a mean expiration of 15 days.

TABLE 1

| N | Donor | MSC | Recipient | Radiation (cGy) | Survival |
|---|---|---|---|---|---|
| 12 | LEW | 5 × 10⁶ | WF | 1000 | 0/12 |
| 12 | LEW | 0 | WF | 1000 | 0/12 |
| 31 | LEW | 5 × 10⁶ | WF | 900 | 21/31 |
| 22 | LEW | 0 | WF | 900 | 0/22 |
| 6 | LEW | 5 × 10⁶ | WF | 500 | 6/6 |
| 6 | LEW | 0 | WF | 500 | 6/6 |
| 6 | LEW | 5 × 10⁶ | WF | 0 | 6/6 |
| 5 | LEW | 0 | WF | 0 | 5/5 |

This treatment regimen was repeated at both higher and lower levels of irradiation. At 1,000 cGy total body irradiation (TBI), the rescue effect was lost with no animals in either the experimental or the control group surviving past 9 days. Without wishing to be bound by theory, this level of radiation is believed to be both lethal and myloablative allowing only minimal marrow constituents to survive post-exposure. At a lower level of 500 cGy, both experimental and control groups experienced no ill effects and survival was 100%. Similarly, control animals receiving 5×10⁶ MSC and no radiation experienced no ill effects and demonstrated a 100% survival rate.

Recovery of Hematopoiesis after 900 cGy+5×10⁶ MSC

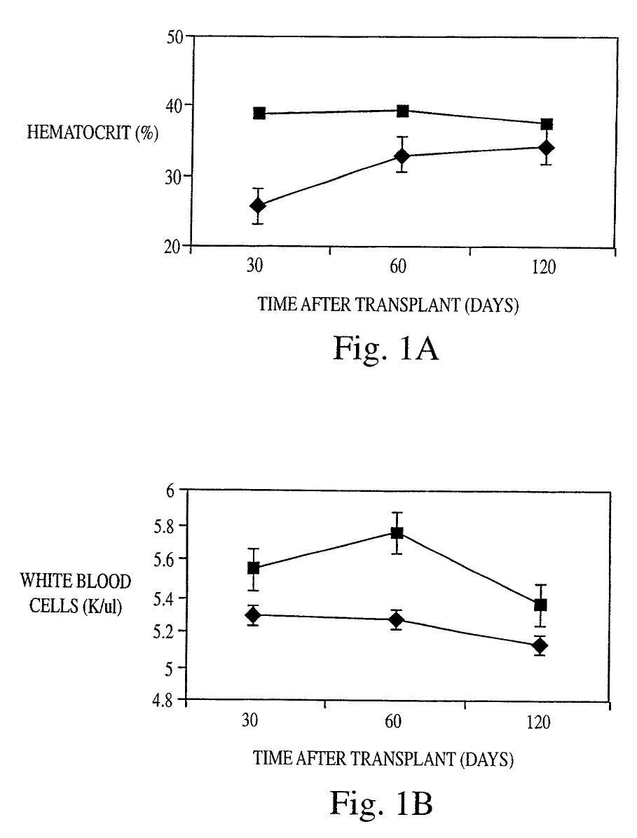

Animals receiving lethal radiation died from profound sepsis and the inibility to mount and maintain an adequate immune response. The severe neutropenia seen early after radiation was subsequently compounded by a steady drop in hematocrit from lack of erythropoiesis. Both leukocyte and erythrocyte recovery was monitored in experimental and control animals at 2, 3 and 4 months (FIG. 1). Five rats in each group had CBCs performed by the clinical laboratory at AUHS. This analysis included hemoglobin, hematocrit, leukocyte count, platelets count, and a manual differential. The hematocrits over time reached levels comparable to controls not receiving radiation (FIG. 1A). All of the irradiated animals were grossly anemic in the immediate post-radiation period with blanching of the ears and paws and loss of retinal hue. However, those animals surviving to 30 days were indistinguishable from untreated littermates by physical examination. Although leukocyte counts did not recover to the same level as controls, adequate leukocyte recovery into the immunocompetent range was noted in all rats analyzed after 30 days (FIG. 1B).

Rescued Animals Exhibit No Signs of GVHD

Rodents reconstituted with whole bone marrow after lethal radiation exhibit many signs of GVHD. Often, this condition, which can be noted by both physical exam and histologic analysis, is associated with very high mortality. Accordingly, all animals receiving allogenic MSCs were examined daily for dermatologic changes, ear erosion, foot pad swelling, weight loss, or diarrhea indicative of GVHD. Upon necropsy, the spleens were weighed, and tissue samples from the small bowel and the tongue were examined microscopically. No animals exhibited gross or microscopic evidence of GVHD.

Irradiated MSC do not Rescue Irradiated Animals

Although MSC have traditionally been demonstrated to possess a high level of radioresistance, the rescue properties of the MSC in these experiments are lost after high dose radiation. Aliquots containing fifty million cells were exposed to 10,000 cGy prior to i.p. injection into irradiated animals. As shown in Table 2, the rescue effect was lost in all but one animal.

TABLE 2

| N | Donor | MSC | Recipient | Radiation (cGy) | Survival |
|---|---|---|---|---|---|
| 12 | LEW | 5 × 10⁶ | WF | 900 | 9/12 |
| 12 | LEW | 5 × 10⁶ | WF | 900 | 1/12 |
| | | Irradiated | | | |
| 12 | LEW | 5 × 10⁶ | WF | 900 | 9/12 |

Endogenous Recovery of Hematopoiesis

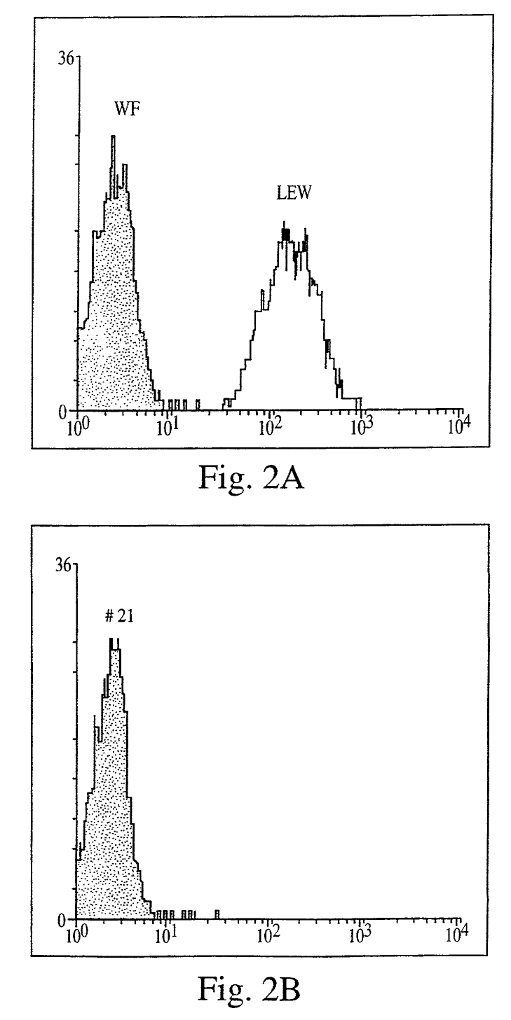

Several reports have demonstrated that complete hemopoietic recovery can take place in the irradiated host by reconstituting with only a few HSC. Thus, possible contamination of WF recipients with donor LEW HSC that may have survived in the MSC cultures and might be a likely explanation for the survival and recovery effect observed was examined. Flow cytometry analysis of PBL demonstrated that no donor LEW cells were present in recipient WF animals. (FIG. 2). Eleven experimental animals and their corresponding untreated controls were bled for peripheral blood 30 days after MSC transplant. The FITC conjugated monoclonal antibody, RTA^{a,b,l}, was used to stain for the LEW MHC-I positive component and a FITC conjugated polyclonal antibody, RTA^{u}, was used for the WF MHC-II positive component. FIG. 2 represents a typical result of the histogram generated by the analysis of PBL from animals treated with 900 cGy+five million MSC after 30 days. FIG. 2A represents the control flow analysis wherein WF and LEW PBL were mixed and stained with RTA^{a,b,l} (MHC-I) clearly demonstrating the delineation of WF and LEW. The strong LEW signal is clearly present after collection of 10,000 events (FIG. 2A). In contrast, no positive LEW staining (RTA^{a,b,l} (MHC-I)) was noted in any of the LEW MSC treated WF recipients as exemplified by recipient rat number 21 (FIG. 2B). These data suggest that contamination with LEW HSC is highly unlikely and that hemopoietic reconstitution in these animals is an endogenous phenomenon.

Real-Time PCR Assay for Male LEW Cells

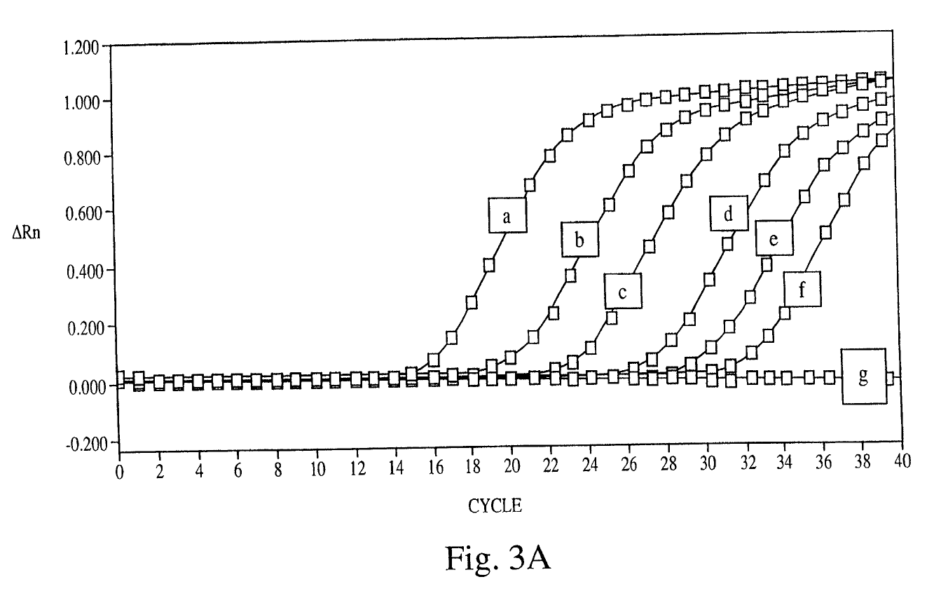

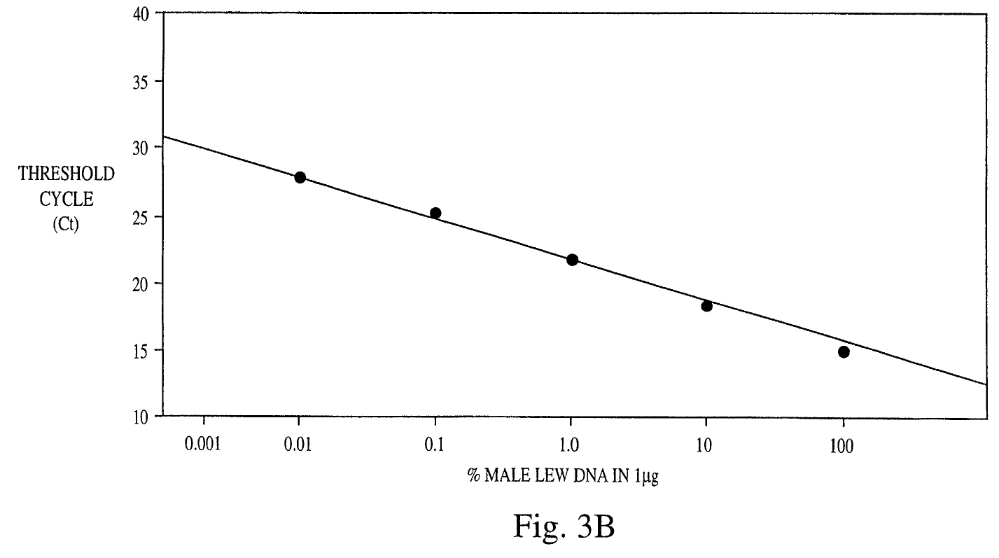

To further demonstrate that hemopoietic reconstitution was endogenous and not caused by donor HSC contamination of the MSC administered to irradiated animals, a highly sensitive real-time PCR quantitative sequence detection assay for the detection of mate rat DNA present in the female host was developed. Using an ABI Model 7700 Real-Time Sequence Detector System from Perkin Elmer (Foster City, Calif.) and Y-chromosome specific PCR primer pairs and Taqman type probes, male DNA was detected in female DNA up to a detection limit of a 1:100,000 dilution of male-to-female DNA or less 10 pg of male DNA present in 1 μg of female DNA (FIG. 3). A set of dilution standards was prepared containing known ratios of male-to-female DNA and the threshold cycle (Ct) (i.e., the cycle number where the level of fluorescent detection reaches an arbitrary threshold value, which in this case was set to be equal to 10 times the standard deviation) was determined for each dilution by plotting the ΔRn (change in detectable fluorescence) as a function of PCR cycle number thus generating an amplification plot for each sample (FIG. 3A). The threshold cycle is correlated to the amount of target nucleic acid being amplified present in a sample. That is, at higher concentrations of target DNA (in this case, rat Y chromosome-specific DNA), the threshold cycle is reached at a lower cycle number. The amplification plots were then used to generate a standard curve of critical threshold (Ct) versus the percentage (%) of male LEW DNA in 1 μg of DNA (FIG. 3B). Using this system, blood, bone, bone marrow, liver, muscle, skin, spleen, and thymus from WF recipients were examined at one and two months after MSC transplantation. Despite the high sensitivity of this assay which is capable of detecting 10 pg of male DNA present in 1 μg of female DNA, no male LEW donor DNA was detected in any of the samples analyzed (Table 3). These data further demonstrate that the hemopoietic recovery in the recipient rats was not due to donor HSC contamination.

TABLE 3

| Tissue | n (1 month) | n (2 months) |
|---|---|---|
| Blood | 6 | 5 |
| Bone | 6 | 5 |
| Bone Marrow | 6 | 5 |
| Liver | 6 | 5 |
| Muscle | 6 | 5 |
| Skin | 6 | 5 |
| Spleen | 6 | 5 |
| Thymus | 6 | 5 |

Lack of Tolerance or Hypersensitization to Solid Organs

Since experimental animals were exposed to both a high level of irradiation and donor antigen, the possibility that donor specific tolerance may have been instituted by this treatment protocol was examined. Four WF recipients at one and two months were given heterotopic vascularized cardiac transplants (Table 4).

TABLE 4

| | | | Re- | | | |
|---|---|---|---|---|---|---|
| | | | cip- | Radiation | Time After | Graft Survival |
| N | Donor | MSC | ient | (cGy) | Transplant | (means in days) |
| 4 | LEW | 5 × 10⁶ | WF | 900 | 2 months | †, †, 4, 9, (6.5) |
| 4 | LEW | 5 × 10⁶ | WF | 900 | 4 months | 7, 8, 10, 12 |
| | | | | | | (9.3) |
| 5 | LEW | 0 | WF | 0 | — | 6, 6, 8, 8,9(7.4) |

†represents technical failures.

† Represents Technical Failures

Two of the four animals in the 2 month group were excluded due to technical error (as indicated by the †). However, the remaining 6 operations were successful with no perioperative complications. No cardiac graft in either the experimental or control groups reached a tolerant state. Of interest is the fact that although tolerance was not demonstrated, neither was hyperacute rejection. Transplanted hearts in the 2 month group had a mean survival of 6.5 days. Hearts in the 4 month group had a mean survival of 9.3 days. These results were not statistically different than control grafts that had a mean survival of 7.4 days.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of rescuing a mammal from a lethal dose of total body irradiation, said method comprising administering marrow stromal cells from an allogenic but otherwise identical donor mammal to an irradiated mammal, thereby rescuing said mammal from a lethal dose of total body irradiation.

2. The method of claim 1, wherein said mammal is selected from the group consisting of a rodent, a horse, a cow, a pig, a dog, a cat, a non-human primate, and a human.

3. The method of claim 2, wherein said mammal is a human.

4. The method of claim 1, wherein said administration is infusion.

5. A method of enhancing hematopoiesis in a mammal, said method comprising administering marrow stromal cells from an allogenic but otherwise identical donor mammal to a mammal, thereby enhancing hematopoiesis in said mammal.

6. The method of claim 5, wherein said mammal is selected from the group consisting of a rodent, a horse, a cow, a pig, a dog, a cat, a non-human primate, and a human.

7. The method of claim 6, wherein said mammal is a human.

8. The method of claim 5, wherein said administration is infusion.

9. A method of enhancing hematopoietic stem cell differentiation in a mammal given a lethal dose of total body irradiation, said method comprising administering marrow stromal cells from an allogenic but otherwise identical donor mammal to an irradiated mammal, thereby enhancing hematopoietic stem cell differentiation in said mammal.

10. The method of claim 9, wherein said mammal is selected from the group consisting of a rodent, a horse, a cow, a pig, a dog, a cat, a non-human primate, and a human.

11. The method of claim 10, wherein said mammal is a human.

12. The method of claim 9, wherein said administration is infusion.

13. A method of enhancing the hematopoietic recovery in a mammal given a lethal dose of total body irradiation, said method comprising administering marrow stromal cells from an allogenic but otherwise identical donor mammal to an irradiated mammal, thereby enhancing the hematopoietic recovery in said mammal.

14. A method of treating a mammal comprising an ablated marrow, said method comprising administering marrow stromal cells from an allogenic but otherwise identical donor mammal to a mammal, thereby treating said mammal comprising an ablated marrow.

15. A method of enhancing hematopoiesis in a mammal comprising an ablated marrow, said method comprising administering marrow stromal cells from an allogenic but otherwise identical donor mammal to a mammal, thereby enhancing hematopoiesis in said mammal comprising an ablated marrow.

16. A method of increasing survival of a mammal exposed to a lethal dose of total body irradiation, said method comprising administering marrow stromal cells from an allogenic but otherwise identical donor mammal to an irradiated mammal, thereby increasing the survival of a mammal exposed to a lethal dose of total body irradiation.
